(19) United States
(12) Patent Application Publication
(10) Pub. No.: US 2023/0242987 A1
(43) Pub. Date: Aug. 3, 2023

(54) METHODS AND KITS FOR EVALUATING THE RISK OF DISEASES OR CONDITIONS ASSOCIATED WITH ATHEROSCLEROSIS

(71) Applicant: National Cheng Kung University, Tainan City (TW)

(72) Inventors: Yen-Wen LIU, Tainan City (TW); Ping-Yen LIU, Tainan City (TW)

(21) Appl. No.: 17/945,832

(22) Filed: Sep. 15, 2022

(30) Foreign Application Priority Data
Sep. 29, 2021 (TW) 110136258

(51) Int. Cl. C12Q 1/6883

(52) U.S. Cl. CPC C12Q 1/6883; C12Q 2600/156; C12Q 2600/112

(57) ABSTRACT

A method and a kit for evaluating the risk of diseases or conditions associated with atherosclerosis by detecting at least one genotype for single nucleotide polymorphism in a biological sample of a subject. The at least one genotype for the single nucleotide polymorphism may be a genotype for rs12657663 in CAMLG gene, a genotype for rs2273970 in GALNT2 gene, a genotype for rs643634 in SPINDOC gene, a genotype for rs737976 in THOC5 gene, or a genotype for rs9988179 in SAMD11 gene.

### CROSS REFERENCE TO RELATED APPLICATIONS

This application claims priority of Taiwan Application No. 110136258, filed on Sep. 29, 2021 under 35 U.S.C. § 119. The entire disclosures of all the above applications are hereby incorporated by reference.

### REFERENCE TO AN ELECTRONIC SEQUENCE LISTING

The contents of the electronic sequence listing (2023-04-20 Sequence Listing XML.xml; Size: 5,670 bytes; and Date of Creation: Apr. 11, 2023) is herein incorporated by reference in its entirety.

### FIELD OF THE INVENTION

The present invention relates to a genetic risk scoring model to identify groups at high risk for developing atherosclerosis-related diseases or conditions. Specifically, the present disclosure relates to methods and kits for evaluating the risk based on the detection of single-nucleotide polymorphisms (SNPs).

### BACKGROUND OF THE INVENTION

Single-nucleotide polymorphism (SNP) is a nucleic acid sequence polymorphism due to a single nucleotide change. That is, the genomes of different individuals may have different nucleotides at the same specific site. The cause of such variation is usually caused by gene deletion, insertion, or substitution.

Among all possible DNA sequence differentiation, SNP is the most common genetic variation. In humans, the probability of occurrence of a SNP is about 0.1%, that is, there may be one SNP in every 1,200 to 1,500 base pairs. About 4 million SNPs have been discovered. On average, there is one SNP for every 1 kb of DNA. In other words, in each person's DNA sequence, at least one “single base variation” occurs in every 1 kb unit length. Due to the high frequency of SNPs, SNPs are often used as genetic markers for related research.

However, not all SNPs have clinical significance. SNPs that have a major impact on disease development and drug therapy are estimated to be only a small fraction of the millions of SNPs. Even if SNPs are generated, it does not necessarily cause changes in amino acid coding or gene expression regulation, or changes in protein structure or activity, and resulting in specific effects on drugs. How to find clinically meaningful functional SNPs from millions of SNPs is a huge challenge.

The oxygen required by the heart mainly depends on the supply of three branched coronary arteries. As long as these blood vessels remain healthy, the heart can maintain full function. When any one of the coronary arteries is narrowed or blocked, it will block the oxygen and nutrient supply to the myocardium, resulting in hypoxia of the heart and inhibiting the contraction of the myocardium, making the heart unable to pump out a normal amount of blood. It may cause heart failure or arrhythmia leading to death.

In recent years, the number of patients with atherosclerotic diseases such as myocardial infarction, stenosis, cerebral apoplexy, peripheral vascular disease, etc. has been found to be increasing due to changes in living habits and aging. For the treatment of such atherosclerotic diseases, percutaneous transluminal angioplasty (PTA) is widely used in clinic. This procedure inserts a metal stent to support the dilated vessel lining Percutaneous transluminal coronary angioplasty (PTCA) is a PTA performed especially for the stenosis or infarction of the coronary arteries of the heart.

However, a proportion of patients will still experience in-stent restenosis for a period of time after PTA. Such patients require repeated procedures of surgery and treatment. Restenosis is a multifactorial process, possibly due to inflammation-induced intimal hyperplasia or smooth muscle hyperplasia and an inflammatory response to atherosclerosis. According to statistics, the probability of in-stent restenosis accounts for about 10% of PCTA. Current treatments for in-stent restenosis include repeat balloon angioplasty, repeat stenting, cutting balloon angioplasty, directional coronary atherectomy, coronary atherectomy, brachytherapy, and drug-eluting stents (DES). Although drug-eluting stents greatly reduce the incidence of in-stent restenosis, “drug-eluting in-stent restenosis” remains a clinical challenge for cardiologists. Current medical technology is still unable to predict patients' response to drug-eluting stents prior to surgery. That is, the risk of developing in-stent restenosis remains unpredictable.

### SUMMARY OF THE INVENTION

One object of the present invention is to provide a genetic risk scoring model to identify groups at high risk for developing atherosclerosis-related diseases or conditions. Specifically, the present disclosure relates to a method and a kit for evaluating the risk based on the detection of single-nucleotide polymorphisms (SNPs). Specifically, the present disclosure estimates the genetic risk of restenosis in drug-eluting stents by calculating the sum of the single nucleotide polymorphisms (SNPs) of five specific genes.

The method for evaluating the risk of diseases or conditions associated with atherosclerosis, comprises the following steps:
a) obtaining a biological sample from an individual;
b) analyzing said sample to determine the genotype of at least one single-nucleotide polymorphism (SNP), wherein the genotype of the at least one single-nucleotide polymorphism is selected from the group consisting of the genotype of rs12657663 in the CAMLG gene, the genotype of rs2273970 in the GALNT2 gene, the genotype of rs643634 in the SPINDOC gene, the genotype of rs737976 in the THOC5 gene, the genotype of rs9988179 in the SAMD11 gene; and
c) determining the risk of the individual suffering from atherosclerosis-related diseases or conditions according to the detected genotype of the single nucleotide polymorphism.

In a preferable embodiment, the SNP rs12657663 is a risk SNP when an allele T at the position of the SNP rs12657663.

In a preferable embodiment, the SNP rs2273970 is a risk SNP when there is an allele A at the position of the SNP rs2273970.

In a preferable embodiment, the SNP rs643634 is a risk SNP when there is an allele C at the position of the SNP rs643634.

In a preferable embodiment, the SNP rs737976 is a risk SNP when there is an allele C at the position of the SNP rs737976.

In a preferable embodiment, the SNP rs9988179 is a risk SNP when there is an allele A at the position of the SNP rs9988179.

In a preferable embodiment, the method further includes detecting the genotypes of a plurality of the single-nucleotide polymorphisms in the biological sample, and summing up the number of risk SNPs, wherein the individual is at increased risk of developing an atherosclerosis-related disease or condition when the sum of the number of risk SNPs is greater than or equal to 3.

In a preferable embodiment, the atherosclerosis-related disease or condition is coronary angiography or computer tomography angiography documented in-stent restenosis, intimal hyperplasia, or coronary arteriosclerosis.

In a preferable embodiment, the in-stent restenosis is drug-eluting in-stent restenosis.

In a preferable embodiment, the biological sample is blood, amniotic fluid, cerebrospinal fluid, tissue fluid, saliva, sweat, urine, fecal matter, skin, or hair.

Another object of the present invention is to provide a kit for evaluating the risk of diseases or conditions associated with atherosclerosis, comprising at least one probe set for detecting the genotype of at least one single-nucleotide polymorphism of the individual, wherein the genotype of the at least one single-nucleotide polymorphism is selected from the group consisting of the genotype of rs12657663 in the CAMLG gene, the genotype of rs2273970 in the GALNT2 gene, the genotype of rs643634 in the SPINDOC gene, the genotype of rs737976 in the THOC5 gene, the genotype of rs9988179 in the SAMD11 gene.

In a preferable embodiment, the probe set includes probes having the nucleotide sequence of at least one of SEQ ID NO: 1 to 5. In other specific embodiments, a probe having the nucleotide sequence of SEQ ID NO: 1 is used to detect the genotype of rs12657663 in the CAMLG gene. A probe having the nucleotide sequence of SEQ ID NO: 2 is used to detect the genotype of rs2273970 in the GALNT2 gene. A probe having the nucleotide sequence of SEQ ID NO: 3 is used to detect the genotype of rs643634 in the SPINDOC gene. A probe having the nucleotide sequence of SEQ ID NO: 4 is used to detect the genotype of rs737976 in the THOC5 gene. A probe having the nucleotide sequence of SEQ ID NO: 5 is used to detect the genotype of the single nucleotide polymorphism rs9988179 in the SAMD11 gene.

### BRIEF DESCRIPTION OF THE DRAWINGS

FIG. 1 is a flow chart of at least one embodiment of the present disclosure, showing the experimental design of a genetic risk score model for in-stent restenosis of drug-eluting stents.

FIG. 2 is a distribution diagram of the result of the genetic risk score model score for in-stent restenosis of drug-eluting stents. When the genetic risk score is higher, the probability of in-stent restenosis in drug-eluting stents is higher.

FIG. 3 shows the association of genetic risk scores with the occurrence of in-stent restenosis in drug-eluting stents after combining the detection results of the five single nucleotide polymorphisms. Wherein, patients with a genetic risk score≥3 were 4.66 times more likely to develop in-stent restenosis than those with a genetic risk score<3.

### DETAILED DESCRIPTION OF THE INVENTION

The detailed description and preferred embodiments of the invention will be set forth in the following content and provided for people skilled in the art to understand the characteristics of the invention.

The present disclosure provides a method and kit for evaluating the risk of diseases or conditions associated with atherosclerosis by detecting SNPs in an individual. There are a lot of SNP information in the existing database, and the sources can be roughly divided into three types, among which the most accounts for about 57%. They are compared through the biological information method EST (expressed sequence tag, which is the cDNA sequence information obtained from a large number of cDNA sequence database clones at one time). In addition, about 42% of the SNP information was generated experimentally, and then obtained by sequence alignment using bioinformatics methods. The final 1% comes from the genome sequencing project.

The EST sequence alignment needs to be carried out by various biological information software, such as PolyBayes, which uses the gene sequence of the human chromosome as the reference sequence, and then performs the alignment between all the EST sequences in the database. After the alignment, we can know which ESTs belong to the same group, and then exclude the possible sequencing errors to obtain SNPs.

Generally, the method of finding SNPs through experiments requires selecting a specific gene or a sequence to be analyzed first, then designing appropriate primers, and then increasing the number of nucleic acid sequences by polymerase chain reaction (PCR). After the number of nucleic acids increases, multiple sequence alignment analysis is performed by computer and software to find possible SNPs.

At present time, large scale of SNP screening by DNA microarray or gene chip can be automated, and it is often used in paternity testing and accurate identification.

The present disclosure provides a method for evaluating the risk based on the detection of single-nucleotide polymorphisms (SNPs), which comprises analyzing said sample to determine the genotype of at least one SNP from a biological sample of an individual and determining the risk of the individual suffering from atherosclerosis-related diseases or conditions according to the detected genotype of the SNP. Wherein, the genotype of the SNP is selected from at least one of the genotype of rs12657663 in the CAMLG gene, the genotype of rs2273970 in the GALNT2 gene, the genotype of rs643634 in the SPINDOC gene, the genotype of rs737976 in the THOC5 gene, and the genotype of rs9988179 in the SAMD11 gene.

As used herein, the terms “individual” and “patient” are used interchangeably and refer to a human or an animal Examples of individuals include but are not limited to humans, monkeys, mice, rats, marmots, ferrets, rabbits, hamsters, cows, horses, pigs, deer, dogs, cats, foxes, wolves, chickens, cassowaries, ostriches, and fish. In embodiments of the present disclosure, the individual is a mammal, which may be a primate, such as a human.

The present disclosure is a method for risk estimation based on an individual's genomic profile. To obtain a genomic profile, an individual's genetic sample can be isolated from the individual's biological sample. In some embodiments of the present disclosure, the biological sample includes a sample from which genetic material (such as RNA and DNA) can be isolated, such as blood, amniotic fluid, cerebrospinal fluid, tissue fluid, saliva, sweat, urine, fecal, skin, hair, and tissues of various body parts.

As used herein, the term “probe” refers to any substance suitable for the specific detection of another substance, such as the allele at the position of the single nucleotide polymorphism rs12657663 in the CAMLG gene. A probe can be an oligonucleotide or a conjugated oligonucleotide that specifically hybridizes to a specific region within an allele of a given SNP. Conjugated oligonucleotides refer to oligonucleotides that are covalently bound to a chromophore or ligand-containing molecule (such as an antigen) and are highly specific for the receptor molecule (such as an antibody specific for an antigen). The probes can also be PCR primers, the primers are used to amplify a specific region within the allele of a given single nucleotide polymorphism. In addition, the probe may be an antibody that specifically recognizes the polypeptide product of the genotype of a given single nucleotide polymorphism or a serological antigen. On the other hand, the probe can be any substance capable of detecting the equivalent genetic marker of the genotype of the specified single nucleotide polymorphism.

The kit provided by the present disclosure further comprises other components such as suitable buffers, genotypes for more accurate detection of a given SNP, reagents for preparing or improving samples, and instructions for performing evaluation methods, etc.

The following Table 1 shows the correlation between the types of alleles and the risk of in-stent restenosis in drug-eluting stents among the genotypes of the SNP s detected by the method of the present disclosure.

TABLE 1

| | | | Other | Risk | | AA | PolyPhen2 | GTEx V6 |
|---|---|---|---|---|---|---|---|---|
| Gene | RSID* | p | Allels | Allele | RAF | change | HDIV score** | tissue*** |
| CAMLG | rs12657663 | 0.0018583 | C | T | 0.22 | V78I | N/A**** | Coronary artery |
| GALNT2 | rs2273970 | 0.0023998 | G | A | 0.34 | V516M | 0.764 | N/A |
| SPINDOC | rs643634 | 0.0046966 | A | C | 0.32 | N/A | N/A | Artery |
| THOC5 | rs737976 | 0.0011858 | T | C | 0.23 | V525I | N/A | Artery |
| SAMD11 | rs9988179 | 0.0020945 | G | A | 0.11 | H78Y | 0.989 | N/A |

*RSID: rapid stain identification series
**PolyPhen2 HDIV: Polymorphism Phenotyping v2 - high-dimensional instrumental variables
***GTEx V6: Genotype-Tissue Expression Project V6
****N/A: non applicable

The clinical data and genetic profiles used in this study were obtained from National Cheng Kung University Hospital, and the study included a total of 2,749 coronary artery patients. In these patients, who had received drug-eluting stents between 2010 and 2019 and were followed for approximately 3 years, 205 of the 2,749 patients had in-stent restenosis of drug-eluting stent. Among the 2,749 patients, only 690 patients were enrolled. After excluding 6 patients with missing clinical information and 54 patients with stage 5D chronic kidney disease, 630 patients were recruited for gene analysis.

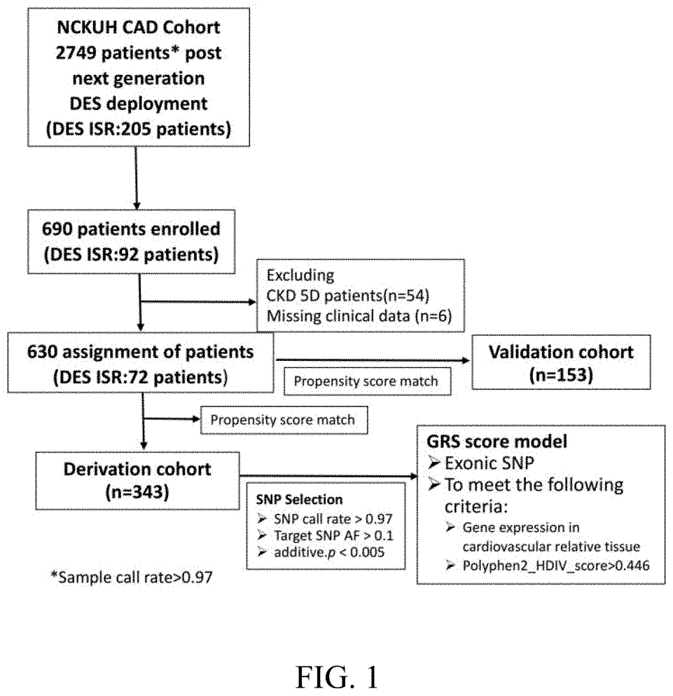

As shown in FIG. 1, 72 of the 630 patients (average age 64.4±10.1 years, male: 80%) suffered from in-stent restenosis of drug-eluting stent. Saliva or blood samples were collected from these 630 patients for genotyping. These participants were categorized into derivation cohort and validation cohort to determine the genetic risk score (GRS) for the possibility of in-stent restenosis of drug-eluting stent. GRS is defined as the sum of the number of SNPs with risk alleles. After propensity score matching, there were 343 and 153 patients in the derivation cohorts and validation cohorts respectively. The corresponding probe sequences designed to detect the genotype of SNPs are shown in Table 2.

TABLE 2

| rsID | chromosome | position | Probe Sequence (5′ → 3′) |
|---|---|---|---|
| rs12657663 | 5 | 134076812 | TCCTGTACTGACTGAATCACCCAGCACTACTCG |
| | | | CTTTGAAACGGAAGGAA (SEQ ID NO: 1) |
| rs2273970 | 1 | 230415148 | ACCTGTGCCTGGACAGTCGCACGGCCAAGAGCG |
| | | | GGGGCCTAAGCGTGGAG (SEQ ID NO: 2) |
| rs643634 | 11 | 63585804 | TCGTCGTTCTCCTTGACTCTGAGGATAACCCAT |
| | | | CCCTCCCTAAAAGGAGC (SEQ ID NO: 3) |
| rs737976 | 22 | 29913272 | GTAAGGGTAACTTGTCACTCCTTCACCTACCAT |
| | | | GTAATCCTCATGGGCAA (SEQ ID NO: 4) |
| rs9988179 | 1 | 865694 | CCTTCCTGTCCGGCCCTGGTCCAAGTACCTTCT |
| | | | CTTCATGATACGGATGT (SEQ ID NO: 5) |

The detailed information of single-nucleotide polymorphism genotypes selected by this research is shown in Table 1. Each genotype will have two alleles, single-nucleotide polymorphisms with risk alleles are defined as risk SNPs and count as 1 point, no risk alleles of single-nucleotide polymorphisms are counted as 0 points.

The score of “genetic risk of in-stent restenosis of drug-eluting stent” can be calculated by summing the scores of risk SNPs in these five risk genes. It can determine an individual's risk of diseases or conditions associated with atherosclerosis, especially the risk of in-stent restenosis of drug-eluting stent.

The results of the selected five SNPs genotype analysis studies showed that the incidence of in stent restenosis of drug-eluting stents in the derivation cohort was 14.58% (50/343), and the incidence of in-stent restenosis of drug-eluting stent in the validation cohort was 16.99% (26/153). The risk classification of the five SNPs selected by the derivation cohort are shown in Table 3. It can be found from Table 3 that, compared with the general alleles of SNPs, the proportion of in-stent restenosis of drug-eluting stent increased significantly when risk alleles were present in selected five SNPs.

TABLE 3

| SNP | rs12657663 | rs2273970 | rs643634 | rs737976 | rs9988179 |
|---|---|---|---|---|---|
| (Gene) | (CAMLG) | (GALNT2) | (SPINDOC) | (THOC5) | (SAMD11) |
| High risk | 22.9%* | 19.5% | 18.8% | 22.7% | 25% |
| (1 or 2 risk allele) | (30/131) | (39/200) | (37/197) | (32/141) | (17/68) |
| Low risk | 9.5% | 7.7% | 9.0% | 8.9% | 12% |
| (no risk allele) | (20/211) | (11/143) | (13/145) | (18/202) | (33/275) |
| odds ratio | 2.83 | 2.90 | 2.34 | 2.99 | 2.44 |
| (95% C.I.) | (1.47 to 5.54) | (1.39 to 6.53) | (1.16 to 5.01) | (1.54 to 5.95) | (1.18 to 4.91) |
| p-value | 0.0008758 | 0.002893 | 0.01287 | 0.0005386 | 0.01148 |

*Percentage of patients with in-stent restenosis (number of cases/total number of genotypes)

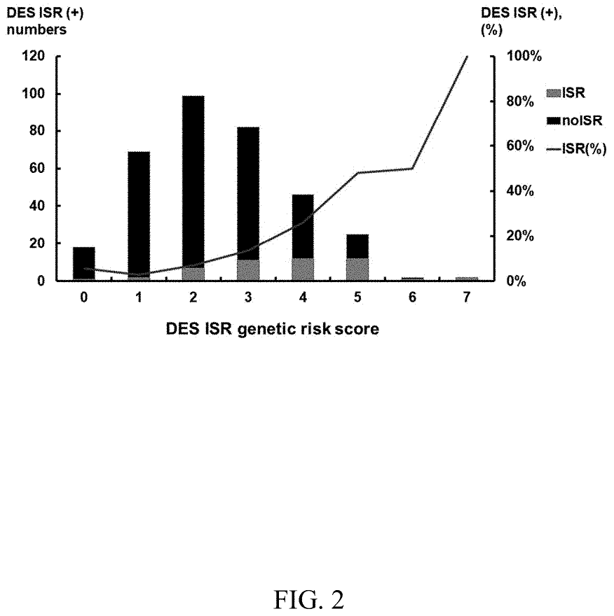

FIG. 2 is the distribution pattern of the genetic risk score of this study. It can be found that when the sum of the genetic risk scores of the selected five SNPs is higher, the probability of in-stent restenosis of drug-eluting stents is higher.

Table 4 shows that after combining the scores of the selected five risk SNPs, the proportion of patients with in-stent restenosis of drug-eluting stent in high-risk categories and low-risk categories for derivation cohort and validation cohort.

TABLE 4

| Merge five risk SNPs | derivation cohort | validation cohort |
|---|---|---|
| High risk | 24.5% | 25.0% |
| (more than 2 risk allele) | (39/157) * | (19/76) |
| Low risk | 5.9% | 9.1% |
| (0 or 1 or 2 risk allele) | (11/186) | (7/77) |
| odds ratio (95% C.I.) | 5.23 | 3.31 |
| | (2.50 to 11.80) | (1.22 to 9.99) |
| p-value | 9.238 × 10⁻⁷ | 0.01004 |

* Percentage of patients with in-stent restenosis (number of cases/total number of genotypes)

From Table 4, when the sum of the number of risk alleles in the selected 5 SNPs≥3 (high risk), the proportion of patients with in-stent restenosis of drug-eluting stent in the derivation cohort and the validation cohort were 24.5% and 25.0% respectively. When the sum of the number of risk alleles in the selected 5 SNPs<3 (low risk), the proportion of patients with in-stent restenosis of drug-eluting stent in the derivation cohort and validation cohort was significantly lower, 5.9% and 9.1% respectively.

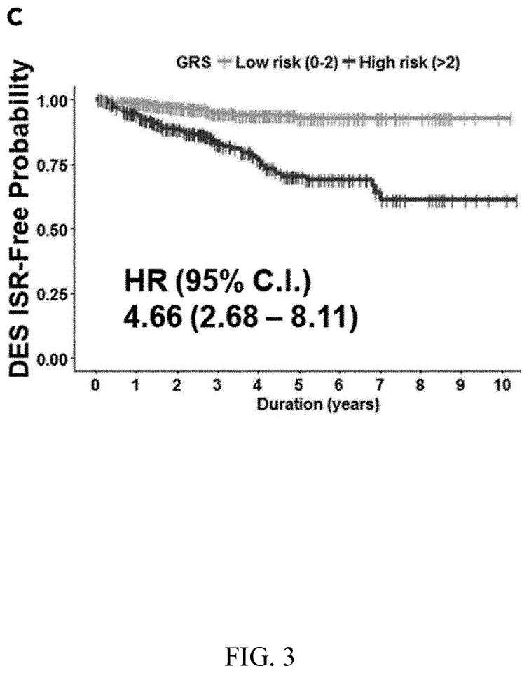

As shown in FIG. 3, it was found that patients with a “genetic risk” score≥3 had a 4.66-fold higher risk of developing “in-stent restenosis of drug-eluting stents” than those with a score<3. That is, the “Genetic Risk Scoring Model” provided by the method of the present disclosure can accurately predict the occurrence of in-stent restenosis of drug-eluting stent. Since the drug-eluting stent is very expensive, the evaluation method of the present disclosure can predict the probability of in-stent restenosis in the future before the percutaneous transluminal angioplasty with drug-eluting stent. It allows patients and cardiologists to have more information to determine the appropriate course of treatment prior to treating coronary artery disease.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

1. A method for evaluating the risk of diseases or conditions associated with atherosclerosis, comprising:
a) obtaining a biological sample from an individual;
b) analyzing said sample to determine the genotype of at least one single-nucleotide polymorphism (SNP), wherein the genotype of the at least one single-nucleotide polymorphism is selected from the group consisting of the genotype of rs12657663 in the CAMLG gene, the genotype of rs2273970 in the GALNT2 gene, the genotype of rs643634 in the SPINDOC gene, the genotype of rs737976 in the THOC5 gene, the genotype of rs9988179 in the SAMD11 gene; and
c) determine the risk of the individual suffering from atherosclerosis-related diseases or conditions according to the detected genotype of the single nucleotide polymorphism.

2. The method as claimed in claim 1, wherein the SNP rs12657663 is a risk SNP when there is an allele T at the position of the SNP rs12657663.

3. The method as claimed in claim 1, wherein the SNP rs2273970 is a risk SNP when there is an allele A at the position of the SNP rs2273970.

4. The method as claimed in claim 1, wherein the SNP rs643634 is a risk SNP when there is an allele C at the position of the SNP rs643634.

5. The method as claimed in claim 1, wherein the SNP rs737976 is a risk SNP when there is an allele C at the position of the SNP rs737976.

6. The method as claimed in claim 1, wherein the SNP rs9988179 is a risk SNP when there is an allele A at the position of the SNP rs9988179.

7. The method as claimed in claim 1, further comprising detecting the genotypes of a plurality of the single-nucleotide polymorphisms in the biological sample, and summing up the number of risk SNPs, wherein the individual is at increased risk of developing an atherosclerosis-related disease or condition when the sum of the number of risk SNPs is greater than or equal to 3.

8. The method as claimed in claim 1, wherein the atherosclerosis-related disease or condition is coronary angiography or computer tomography angiography documented in-stent restenosis, intimal hyperplasia, or coronary arteriosclerosis.

9. The method as claimed in claim 8, wherein the in-stent restenosis is drug-eluting in-stent restenosis.

10. The method as claimed in claim 1, wherein the biological sample is blood, amniotic fluid, cerebrospinal fluid, tissue fluid, saliva, sweat, urine, fecal matter, skin, or hair.

11. A kit for evaluating the risk of diseases or conditions associated with atherosclerosis, comprising at least one probe set for detecting the genotype of at least one single-nucleotide polymorphism of the individual, wherein the genotype of the at least one single-nucleotide polymorphism is selected from the group consisting of the nucleotide of rs12657663 in the CAMLG gene, the nucleotide of rs2273970 in the GALNT2 gene, the nucleotide of rs643634 in the SPINDOC gene, the nucleotide of rs737976 in the THOC5 gene, the nucleotide of rs9988179 in the SAMD11 gene.

12. The kit as claimed in claim 11, wherein the at least one probe set includes a probe having the nucleotide sequence of at least one of SEQ ID NO: 1 to 5.


SEQUENCE LISTING (5 sequences)

SEQ ID NO: 1 - DNA, 50 bp, synthetic construct
  source 1..50: mol_type = other DNA
tcctgtactgactgaatcacccagcactactcgctttgaaacggaaggaa

SEQ ID NO: 2 - DNA, 50 bp, synthetic construct
  source 1..50: mol_type = other DNA
acctgtgcctggacagtcgcacggccaagagcgggggcctaagcgtggag

SEQ ID NO: 3 - DNA, 50 bp, synthetic construct
  source 1..50: mol_type = other DNA
tcgtcgttctccttgactctgaggataacccatccctccctaaaaggagc

SEQ ID NO: 4 - DNA, 50 bp, synthetic construct
  source 1..50: mol_type = other DNA
gtaagggtaacttgtcactccttcacctaccatgtaatcctcatgggcaa

SEQ ID NO: 5 - DNA, 50 bp, synthetic construct
  source 1..50: mol_type = other DNA
ccttcctgtccggccctggtccaagtaccttctcttcatgatacggatgt